(19) United States
(12) Patent Application Publication
(10) Pub. No.: US 2015/0366648 A1
(43) Pub. Date: Dec. 24, 2015

(54) BLOOD FILTER WITH SCAFFOLDS CONFIGURED FOR EXTENDED IMPLANTED DURATION BEFORE RETRIEVAL

(71) Applicant: Cordis Corporation, Fremont, CA (US)

(72) Inventor: Gerhard Johan Golden, Dublin, CA (US)

(73) Assignee: CORDIS CORPORATION, Fremont, CA (US)

(21) Appl. No.: 14/310,446

(22) Filed: Jun. 20, 2014

(51) Int. Cl. A61F 2/01

(52) U.S. Cl. CPC A61F 2/01; A61F 2002/011; A61F 2230/0093; A61F 2230/0078

(57) ABSTRACT

Various embodiments for a blood filter are described. In particular, the filter is provided with scaffolds in the main body that are spaced at a predetermined distance away from tissues in which the filter is implanted to allow for retrievability beyond the known or recommended duration. Other features are also described and shown to allow for attainment of the extended duration and retrievability.

### BACKGROUND

Some basic types of medical filters are generally known, wherein a single filter element, mesh or member extends across the direction of flow inside a blood vessel. Several features are desirable for medical filters, including non-surgical or percutaneous delivery of the filter to a desired site, and expansion from a preferably small initial size to an expanded working size that matches the vascular anatomy at the desired site. Also, a medical filter should of course preferably capture a sufficient percentage of thrombus, while allowing blood to flow freely through the filter.

Another desirable feature is a capability to remain in the desired position for treatment through a period of time, and also to offer the physician the option during that time of leaving the filter in place permanently, or retrieving the filter when no longer needed.

In addition, a medical filter should preferably have a design whereby the filter is stable in the vessel, such that the filter has little or no tendency to “tilt” and may become less effective in capturing thrombus. Some medical filters may be used in the vena cava, and may be described in such event as a “vena cava filter.”

Prior medical filters may consist of a network of interconnected ribs, which extend substantially in a radial direction in relation to the blood vessel. Unfortunately, an entire filter may shift position if one of the ribs were to break. In addition, the free ends of the ribs, which may be positioned under a certain pressure against the internal wall of the blood vessel, may cause trauma to the vessel wall, or may become embedded within.

A disadvantage of some known medical filters may be a possibility of shifting position or tilting inside the blood vessel, even when the filter maintains its proper shape, if a prior filter may have been incorrectly placed in a blood vessel which is too wide. In such an event, a medical filter may not grab sufficient hold on the internal wall of the blood vessel.

A medical filter may be delivered through a catheter in a compressed shape, where it tends to resiliently expand within the blood vessel. The medical filter may tend to trap thrombus or particles, and resist their movement further downstream. The filter may include, in a position of use, an outer shape corresponding to the internal diameter of the blood vessel transverse to the longitudinal direction hereof.

A medical filter which may also be implanted permanently or temporarily. It is preferable to implant the filter initially without deciding at that time whether the filter will eventually be retrieved or is to remain permanently. It is also desirable that if the filter is retrieved, then it should be retrieved as easily as possible.

To help in successful retrieval, a desirable factor is to avoid endothelialization or in-growth of the vessel wall around the structural members of the filter.

On a retrievable filter, it is also desirable to provide releasable temporary position stabilizers, to resist a possibility of tilting and to enhance position retention.

### SUMMARY OF THE DISCLOSURE

Recognizing certain disadvantages of the known filter, I have, in one aspect, devised a blood filter blood filter that includes: a first collar disposed on a longitudinal axis extending through the filter; a first plurality of scaffold members (or ribs) extending from the collar away from the longitudinal axis to define a first generally conic-like dome about the longitudinal axis with a maximal first diameter of the first generally conic-like dome when the blood filter is deployed; a second plurality of scaffold members (or ribs) connected to the first plurality of scaffold members, the second plurality of scaffold members extending generally parallel to the longitudinal axis to define a cylinder about the longitudinal axis with a second diameter approximately ⅔ that of the maximal diameter of the first generally conic-like dome; and a third plurality of scaffold members (or ribs) connected to the second plurality of scaffold members, the third plurality of scaffold members extending towards a second collar disposed on the longitudinal axis to define a second generally conic-like dome about the longitudinal axis and with a maximal third diameter.

Other features of my blood filter designs are also provided in different configurations or permutations. For example, first plurality of scaffold members may include six scaffold members extending away from the longitudinal axis and each of the six scaffold members bifurcating into two scaffold members that join together at a predetermined radius with respect to the longitudinal axis; the third plurality of scaffold members may include six scaffold members extending away from the longitudinal axis and each of the six scaffold members bifurcating into two scaffold members that join together at a predetermined radius with respect to the longitudinal axis; a barb is formed proximate the location where the two scaffold members join together so that six barbs are configured to extend along the longitudinal axis and disposed about the longitudinal axis proximate the maximal diameter of the second generally conic-like dome; the first collar includes a hook so that the blood filter is retrievable with a snare after implantation into a blood vessel; the second collar includes a hook so that the blood filter is retrievable with a snare after implantation into a blood vessel; the first, second and third plurality of scaffold members comprise at least a shape memory material; or the shape memory material may include nitinol.

These and other embodiments, features and advantages will become apparent to those skilled in the art when taken with reference to the following more detailed description of the exemplary embodiments of the invention in conjunction with the accompanying drawings that are first briefly described.

### BRIEF DESCRIPTION OF DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements), in which:

FIG. 1A shows a perspective view of a filter arranged according to the principles of the present invention, in an expanded shape;

FIG. 1B shows a perspective view of another embodiment of a filter arranged according to the principles of the present invention, in an expanded shape;

FIG. 1C illustrates a sectional view of the filter in a fully expanded configuration;

FIG. 1D illustrates a perspective view of the virtual surfaces F1, CYL, and F2 as defined by the scaffolds;

FIG. 2 illustrates an end elevational view along a longitudinal axis of a filter according to the principles of the present invention, in an expanded configuration;

FIGS. 3 and 4 show partial elevation views of a hook structure of a filter according to the principles of the present invention;

FIGS. 5-8 show partial views of shoulder portions of a medical filter, according to the principles of an embodiment of the present invention;

FIG. 9 illustrates a sectional view of the filter implanted in a native vessel;

FIGS. 10 and 11 illustrate respective steps involved in retrieval of the filter.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the term “bio-resorbable” includes a suitable biocompatible material, mixture of materials or partial components of materials being degraded into other generally non-toxic materials by an agent present in biological tissue (i.e., being bio-degradable via a suitable mechanism, such as, for example, hydrolysis) or being removed by cellular activity (i.e., bioresorption, bioabsorption, or bioresorbable), by bulk or surface degradation (i.e., bioerosion such as, for example, by utilizing a water insoluble polymer that is soluble in water upon contact with biological tissue or fluid), or a combination of one or more of the bio-degradable, bio-erodable, or bio-resorbable material noted above. Examples of suitable polymeric compounds include polymers from the group including cellulose, collagen, albumin, casein, polysaccharides (PSAC), polylactide (PLA), poly-L-lactide (PLLA), polyglycol (PGA), poly-D,L-lactide-co-glycolide (PDLLA-PGA), polyhydroxybutyric acid (PHB), polyhydroxyvaleric acid (PHV), polyalkyl carbonates, polyortho esters, polyethylene terephtalate (PET), polymalonic acid (PML), polyanhydrides, polyphosphazenes, polyamino acids, and the copolymers thereof, as well as hyaluronic acid. Metallic biodegradable materials are primarily based on alloys of magnesium, iron, or tungsten.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

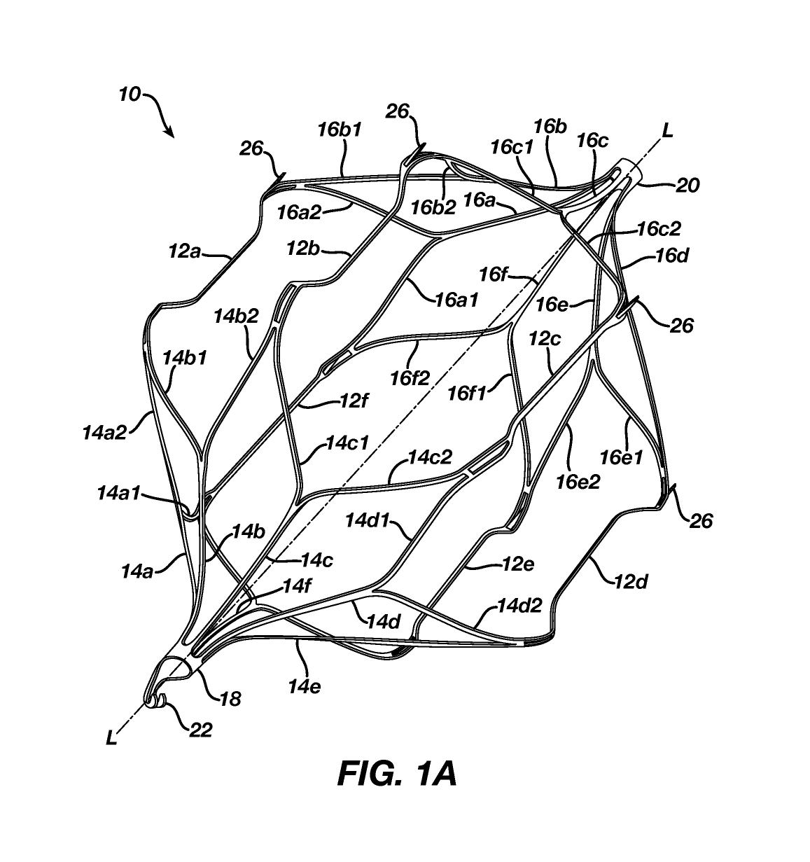

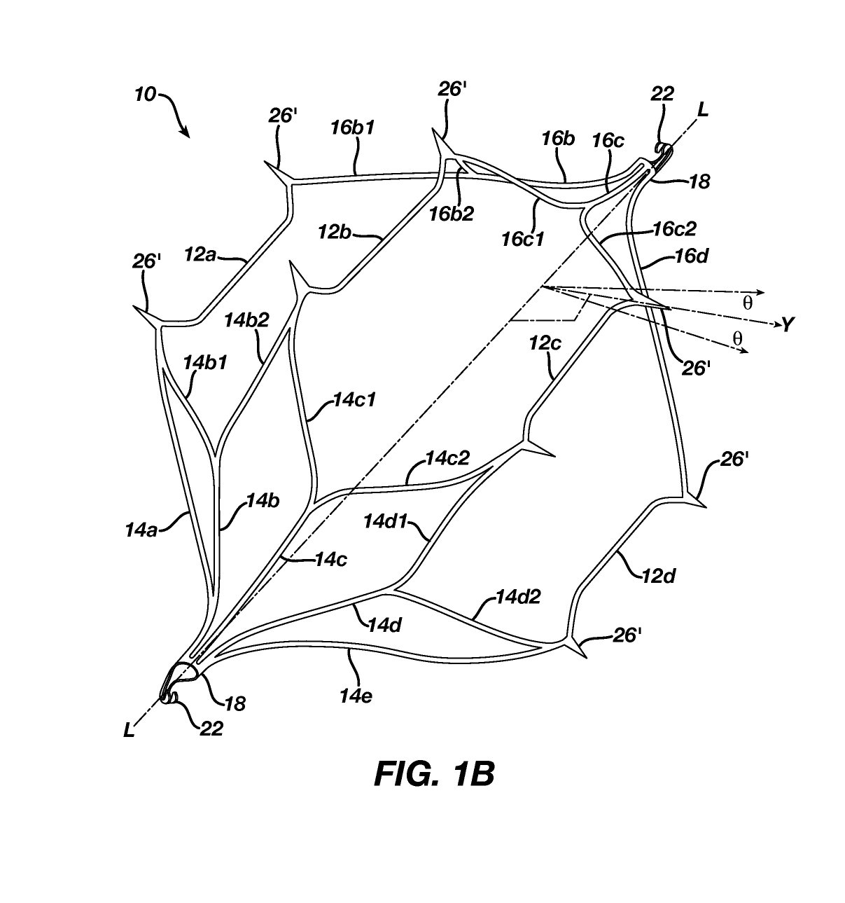

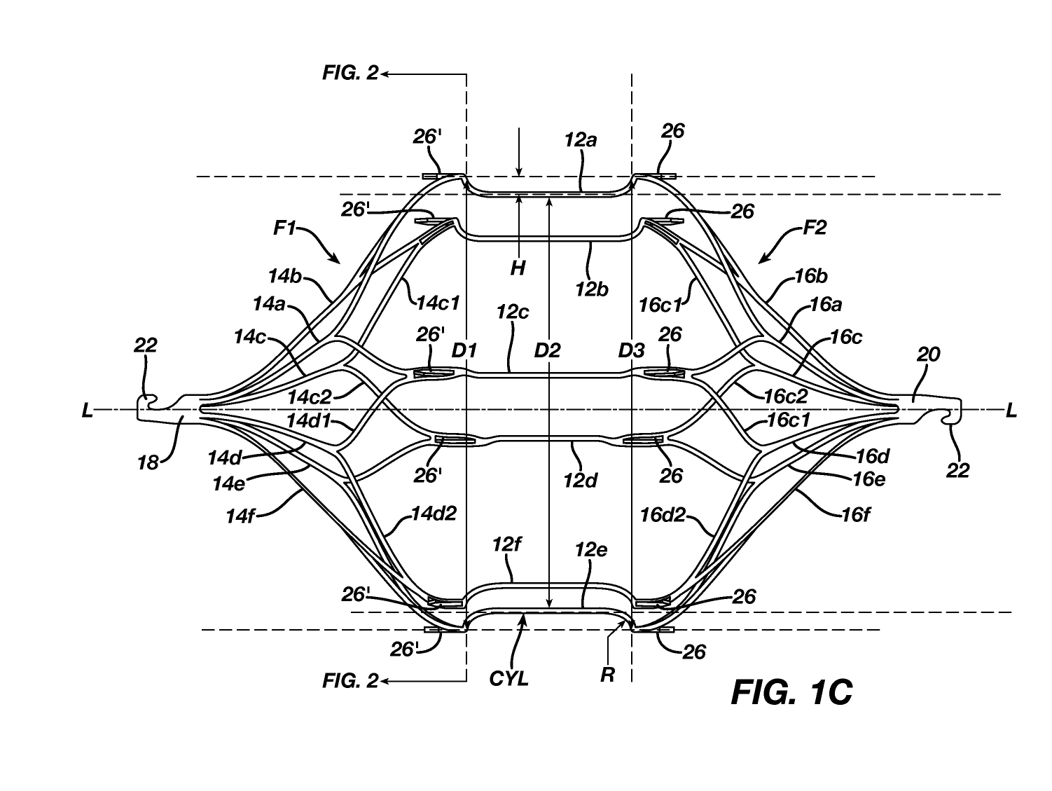

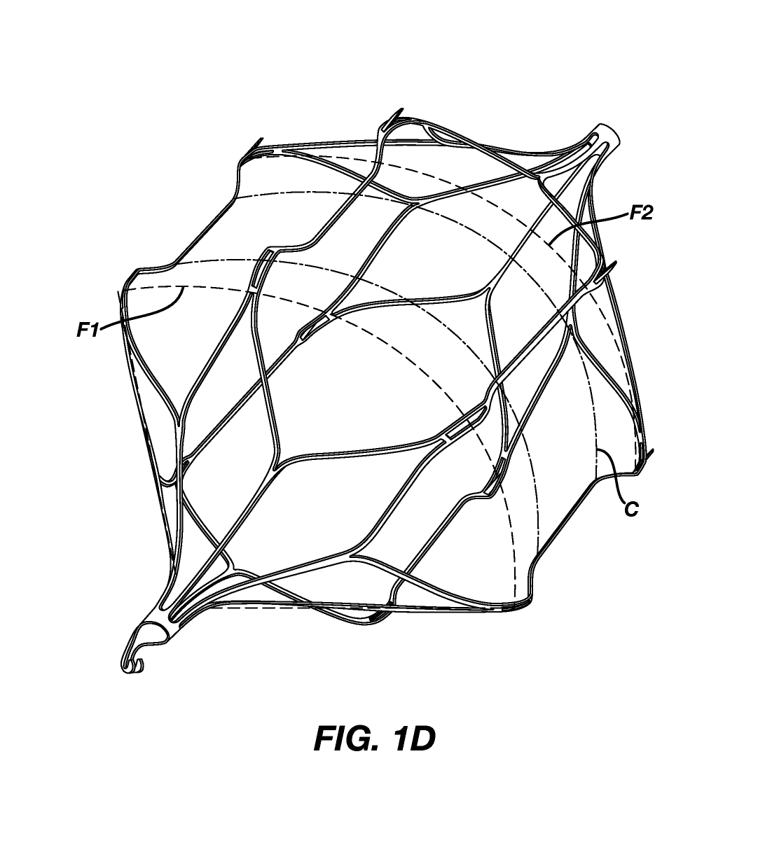

Referring now to the figures wherein like numerals indicate the same element throughout the views, there is shown in FIGS. 1A-C and 2-9 a retrievable blood filter.

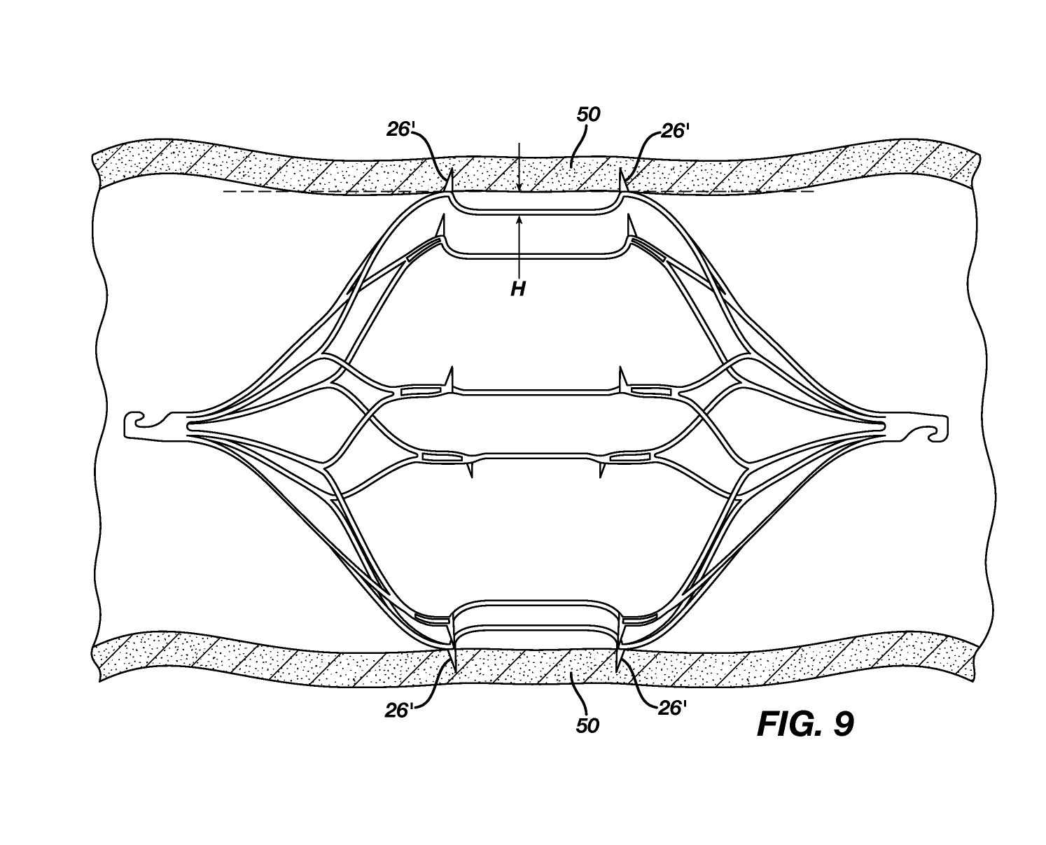

A medical filter according to the present invention is illustrated in FIGS. 1-9. The filter 10 is preferably made of a resilient material, and tends to expand from an initial compressed shape to an expanded shape, as depicted diagrammatically in FIG. 9.

In the expanded shape, the filter 10 preferably has a series of longitudinal ribs 12, aligned essentially parallel with a longitudinal axis of the filter 10. A plurality of members preferably define a first and second filter section 14 and 16, arranged near a retrieval end of the filter and an insertion end, respectively. A pair of central collars 18 and 20 is also preferably positioned at the retrieval end and the insertion end, respectively. A hook structure 22 is attached to the retrieval collar 18.

In viewing FIG. 1A, it can be seen that the filter 10 has a longitudinal axis L-L extending through the center of the filter 10. At one end of the filter, a collar 18 is disposed on the longitudinal axis L-L with a first plurality of scaffold members (14a, 14b, 14c, 14d, 14e, 14f, . . . 14n (where n=any suitable integer)) extending or flaring away from the longitudinal axis (at a given slope) so as to define a virtual dome like or even a frustoconic-like dome F1 (FIG. 1D). It should be noted that each of the plurality of primary scaffold members 14a-14f bifurcates into two secondary scaffold members that has its slope approaching zero with respect to axis L-L at their maximal first diameter D1 (FIG. 1B) to define a base of the virtual dome F1. By way of example, primary scaffold member 14c bifurcates into secondary scaffold members 14c1 and 14c2 which form a curvilinear path with generally zero slope at the maximal first diameter D1 to define the base of the virtual generally frustoconic-like dome F1. In general, the first plurality of primary scaffold members preferably have six scaffold members (14a-14f) extending away from the longitudinal axis L-L and each of the six scaffold members (14a-14f) bifurcates into two secondary scaffold members (14a1, 14a2, 14b1, 14b2, 14c1, 14c2, 14d1, 14d2, 14e1, 14e2, 14f1, 14f2) that join together at a predetermined radius (or ½ D1 in FIG. 1B) with respect to the longitudinal axis L-L. In the preferred embodiment, the maximum radius of the base of the first virtual conic surface F1 is about 15 millimeters (or D1˜30 mm).

Continuing with FIG. 1A, a second plurality of scaffold members (12a, 12b, 12c, 12d, 12e, 12f . . . 12n) are connected to the first plurality of scaffold members 14a-14f. In contrast to the flaring of the first plurality of scaffold members, the second plurality of scaffold members extend generally parallel to the longitudinal axis to define a cylinder C (FIG. 1D) about the longitudinal axis L-L. Of note in FIG. 1B is that the second diameter D2 for the virtual cylinder CYL is approximately ⅔ that of the maximal first diameter D1 of the first generally conic-like dome F1 to provide for a separation distance H between the first diameter D1 and the second diameter D2. A third plurality of primary scaffold members (16a, 16b, 16c . . . 16n) are connected to the second plurality of scaffold members (12a, 12b, 12c . . . 12n) via respective bifurcations (16a1, 16a2, 16b1, 16b2, 16c1, 16c2 and so on) of the third plurality of primary scaffold members (16a, 16b, 16c . . . ). The third plurality of scaffold members (16a, 16b, 16c . . . 16n) including the respective bifurcations (16a1 and 16a2, 16b1, and 16b2, 16c1 and 16c2 . . . ) define a virtual dome-like structure, which can also be viewed as a second virtual frustoconic F2 in FIG. 1D. In general, the third plurality of scaffold members preferably have six primary scaffold members (16a-16f) extending away from the longitudinal axis L-L and each of the six scaffold members (16a-16f) bifurcates into two secondary scaffold members (16a1, 16a2, 16b1, 16b2, 16c1, 16c2, 16d1, 16d2, 16e1, 16e2, 16f1, 16f2) that join together at a predetermined radius (where predetermined radius ˜½ D2 in FIG. 1B) with respect to the longitudinal axis L-L. In the preferred embodiment, the maximum radius of the virtual cylinder CYL is about 10 millimeters (or D2˜20 mm).

The first diameter D1 is preferably equal to third diameter D3 but can be unequal. It should be noted that the second scaffold members 12 can be formed with a connector portion R with a curved configuration with a predetermined radius of curvature.

Taken as a whole, the filter 10 can be represented in FIG. 1D as a skeleton that defines a first conic like virtual dome F1 contiguous with a virtual cylinder CYL which is then contiguous with a second conic like virtual dome F2.

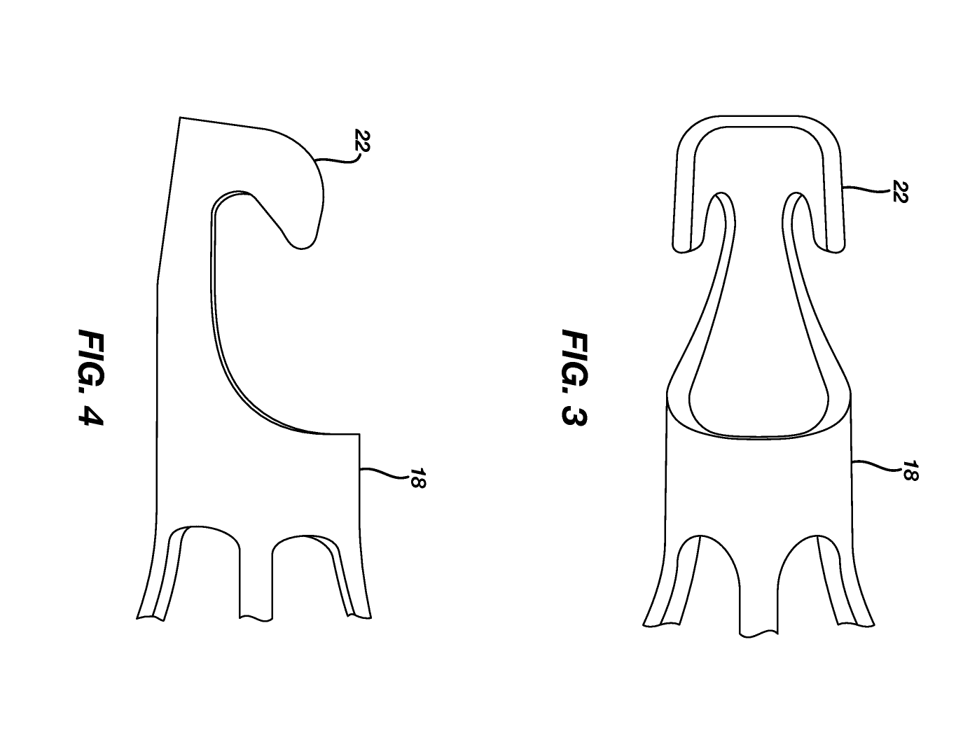

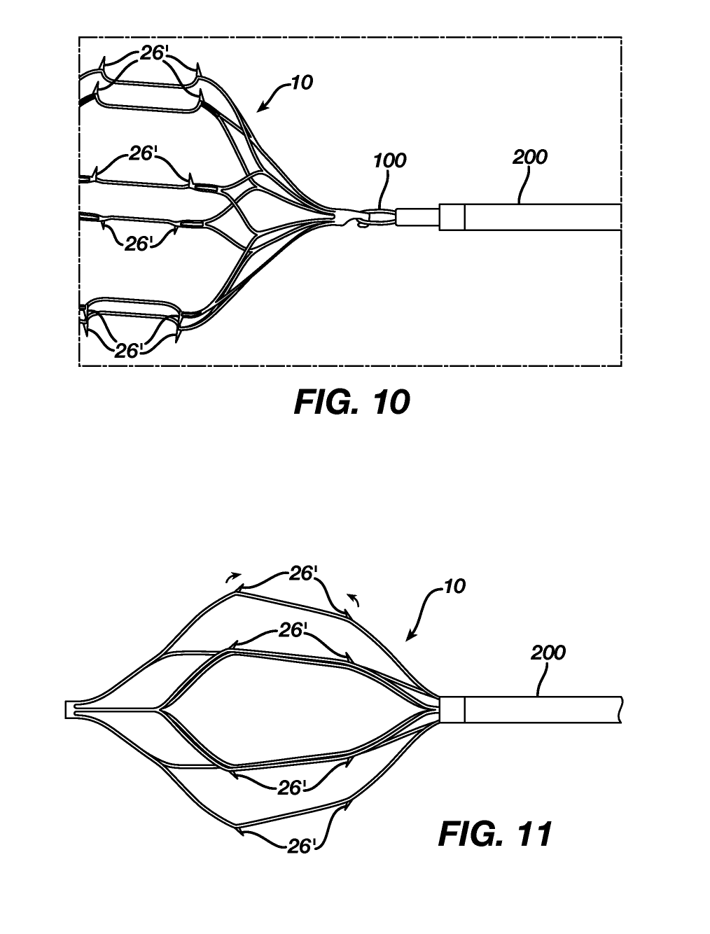

The hook structure 22 of filter 10 may have a T-shape, as shown in FIGS. 3 and 4, with twin hooks 22. It is believed that the twin hook structure 22 may improve the ease of retrieving the filter 10 after implantation of the barbs 26 (or 26′) into biological tissue 50, shown here in FIG. 9. The hook 22 can be disposed on one collar 18 as shown in FIG. 1A. However, to allow for retrieval of the filter 10 with a snare 100 (mounted inside a catheter 200 shown in FIGS. 10 and 11) from either femoral artery or jugular arteries, a second hook 22 can be provided on the second collar 20, shown here in FIGS. 1B and 1C.

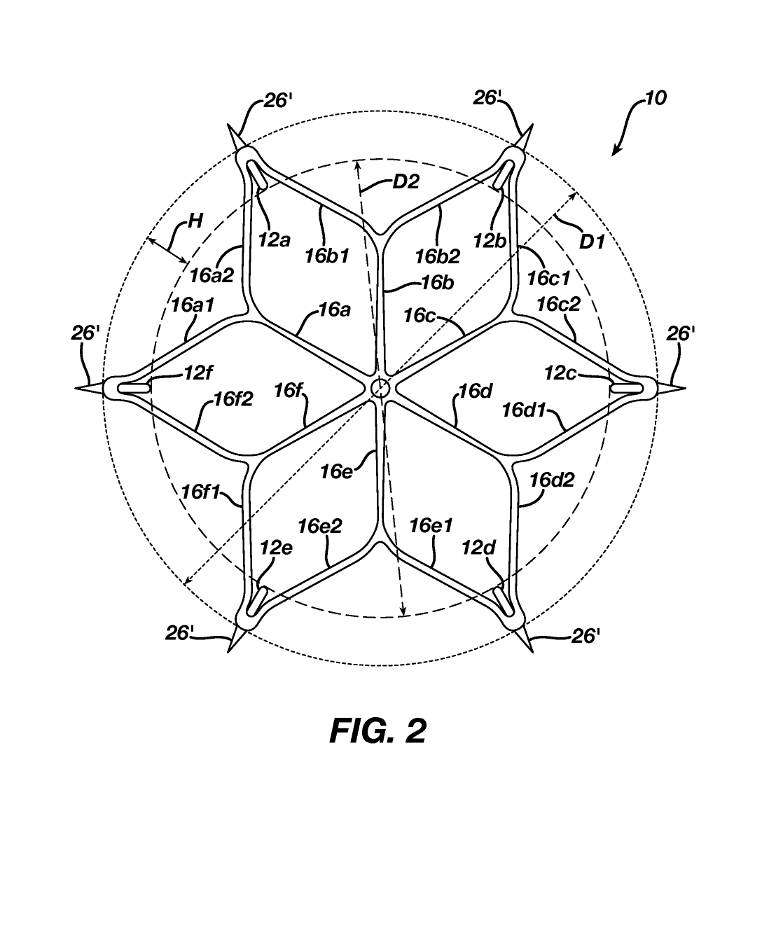

The barbs or anchors 26 extend in a longitudinal direction whereas barbs 26′ extend generally in a direction Y orthogonal to the longitudinal axis L-L, shown here in FIG. 1C. Barbs 26′ can be oriented with a tolerance of θ degrees with reference to the orthogonal axis Y where θ is from about 10 degrees to 30 degrees. As shown in the cross-sectional view of FIG. 2, the barbs 26′ are oriented radially with respect to the longitudinal axis (extending out through the drawing). This arrangement of the barbs 26′ allows, in part, for the benefit of retrieving the filter 10 from either the femoral artery or the jugular artery. This is because when the filter 10 is pulled into sheath 200 by a snare 100 (FIGS. 10 and 11), the barbs 26′ tend to fold in a direction (denoted by arrow in FIG. 10) along the longitudinal axis L-L to present a smaller profile for insertion into the sheath 200.

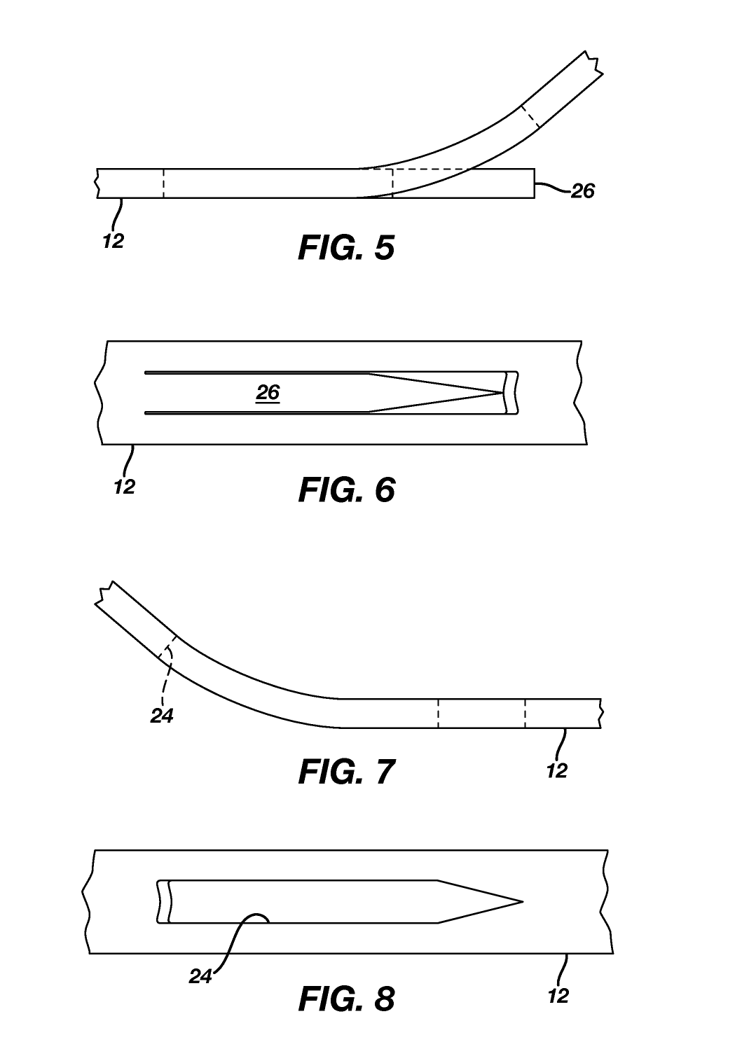

The barbs (26 or 26′) are exposed when the filter 10 is in an expanded shape. As shown in FIGS. 1A, 1C and 5, the anchors 26 and the second filter section 16 define an acute angle. They may be formed as shown in FIGS. 5 and 6, in which a series of cuts in the ribs 12 both shape the anchor 26 and define an aperture for the anchor 26 when the filter is in a compressed shape. The barbs 26 (along with barb 26′) can be formed from a biodegradable material to facilitate removal and retrievability. That is, after a predetermined time period, the barbs 26 (or 26′) are resorbed thereby allowing the filter to be retrieved with minimal trauma or injury to the native vessel in which the filter is implanted in.

The apertures 24 may tend to balance stresses in the filter. In other words, anchors 26 (or 26′) may be formed by cutting them out of the scaffold members or ribs, which will tend to bend and define the center section 15 and the filter section 16. Similarly, the presence of apertures 24 may tend to balance stresses in the ribs, causing them to bend in a complementary manner and define the center section 12 and the filter sections 14 and 16.

The filter 10 is preferably delivered to a desired site for treatment by a delivery catheter, which preferably defines a lumen extending between a proximal hub having a hemostatic valve (not shown) and a distal lumen opening. The filter 10 is preferably initially packaged in a compressed state in a filter cartridge. In use, delivery catheter is inserted along a body passage in a patient until distal end is near a desired site for treatment. Additional details can be found in U.S. Pat. No. 6,989,021 which is hereby incorporated by reference as if set forth in full herein this application.

Medical filters according to the present invention may be made of any suitable material using a variety of methods. One material having the desired characteristics of strength, resilience, flexibility, biocompatibility and endurance is nitinol. Other materials having the desired characteristics may be used as long as such materials are biocompatible, such as, for example, stainless steel, cobalt chromium, thin-film nitinol (e.g., vapor deposited thin-film nitinol). Likewise, the manufacturing methods may include providing a tube, and then cutting a pattern into the tube to enable expansion into the desired shape. Alternatively, wires can be formed instead of the scaffolds formed by laser cutting of a tubular structure. Of course, various other methods are possible, including forming the filter of discrete members and then joining or connecting the members by welding.

In addition to the nitinol mentioned so far, many other materials may also be used for manufacturing a medical filter according to the present invention. By way of alternative, various metals may for instance be used, in which case it is essential that the medical filter assumes the intended shape hereof after having been ejected from the catheter for the purpose of introduction hereof. The medical filter, during introduction, is of course kept in a compressed state, by the catheter. To this end, a configuration may be used decompressing the filter metal due to the elastic properties hereof.

In the axial view, the filter sections on either side of the ribs of the medical filters according to the present invention described above display diamond or polygon shapes. It is also possible to suffice with medical filters of which the filter sections display in axial view (FIG. 3) a star shape, or any other suitable shape, as long as they intercept blood clots or thrombus successfully. An advantage of this feature is that, after passing the first filter section and the tubular section or the elongated body member, a second chance at interception in the form of an additional filter section has been provided. Also, other shapes of the filter sections in axial view are possible, which shapes will occur to those skilled in the field after reading the present description. The shapes of the filter sections in axial view need not be symmetrical, and may have in principle any suitable appearance.

By virtues of this design that I have described and illustrated herein, this filter overcomes the following less than desirable outcomes during a removal after a greater than recommended duration of filter implantation: (a) the filter outermost scaffolds become embedded into the tissue 50 and could prevent filter dislodgment from the tissue; and (b) removal of the known filter may cause rupture of the vessel. Specifically, my filter specifically places the scaffold 12 away from the tissues of the vessel in which my filter can be implanted (FIG. 9). I have configured a separation distance H (FIGS. 1B and 9) to be from about 3 millimeters to about 10 millimeters with 5 millimeters being preferred. By having this separation distance, retrieval of the filter beyond the known (or recommended) implantation period can be achieved. Furthermore, by having barbs that extend in a generally orthogonal direction, the barbs tend to separate from the body tissue with little or no snagging when the filter is collapsed into a smaller configuration during the retrieval process (FIGS. 10 and 11).

While the invention has been described in terms of particular variations and illustrative figures, those of ordinary skill in the art will recognize that the invention is not limited to the variations or figures described. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well.

What is claimed is:

1. A blood filter comprising:
a first collar disposed on a longitudinal axis extending through the filter;
a first plurality of scaffold members extending from the collar away from the longitudinal axis to define a first generally conic-like dome about the longitudinal axis with a maximal first diameter of the first generally conic-like dome when the blood filter is deployed;
a second plurality of scaffold members connected to the first plurality of scaffold members, the second plurality of scaffold members extending generally parallel to the longitudinal axis to define a cylinder about the longitudinal axis with a second diameter approximately ⅔ that of the maximal diameter of the first generally conic-like dome; and
a third plurality of scaffold members connected to the second plurality of scaffold members, the third plurality of scaffold members extending towards a second collar disposed on the longitudinal axis to define a second generally conic-like dome about the longitudinal axis and with a maximal third diameter.

2. The blood filter of claim 1, in which the first plurality of scaffold members may include six scaffold members extending away from the longitudinal axis and each of the six scaffold members bifurcating into two scaffold members that join together at a predetermined radius with respect to the longitudinal axis.

3. The blood filter of claim 1, in which the third plurality of scaffold members may include six scaffold members extending away from the longitudinal axis and each of the six scaffold members bifurcating into two scaffold members that join together at a predetermined radius with respect to the longitudinal axis.

4. The blood filter of claim 2 or claim 3, in which a barb is formed proximate the location where the two scaffold members join together so that six barbs are configured to extend along the longitudinal axis and disposed about the longitudinal axis proximate the maximal diameter of the second generally conic-like dome.

5. The blood filter of claim 1 in which the first collar includes a hook so that the blood filter is retrievable with a snare after implantation into a blood vessel.

6. The second collar includes a hook so that the blood filter is retrievable with a snare after implantation into a blood vessel.

7. The blood filter of claim 1, in which the first, second and third plurality of scaffold members comprise at least a shape memory material.

8. The blood filter of claim 7, in which the shape memory material may include nitinol.

9. A blood filter comprising:
a first collar disposed on a longitudinal axis extending through the filter;
a first plurality of scaffold members extending from the collar away from the longitudinal axis to define a first generally conic-like dome about the longitudinal axis with a maximal first diameter of the first generally conic-like dome when the blood filter is deployed;
a second plurality of scaffold members connected to the first plurality of scaffold members, the second plurality of scaffold members extending generally parallel to the longitudinal axis to define a cylinder about the longitudinal axis with a second diameter approximately ⅔ that of the maximal diameter of the first generally conic-like dome;
a third plurality of scaffold members connected to the second plurality of scaffold members, the third plurality of scaffold members extending towards a second collar disposed on the longitudinal axis to define a second generally conic-like dome about the longitudinal axis and with a maximal third diameter;
a fourth collar disposed on the longitudinal axis and attached to the plurality of the third scaffold members; and
a plurality of barbs disposed on one of the first and third plurality of scaffold member and configured to extend generally along a direction orthogonal to the longitudinal axis.

10. The filter of claim 9, in which the plurality of barbs are disposed on both the first and third plurality of scaffolds.

11. The blood filter of claim 9, in which the first plurality of scaffold members may include six scaffold members extending away from the longitudinal axis and each of the six scaffold members bifurcating into two scaffold members that join together at a predetermined radius with respect to the longitudinal axis.

12. The blood filter of claim 9, in which the third plurality of scaffold members may include six scaffold members extending away from the longitudinal axis and each of the six scaffold members bifurcating into two scaffold members that join together at a predetermined radius with respect to the longitudinal axis.

13. The blood filter of claim 11 or claim 12, in which a barb is formed proximate the location where the two scaffold members join together so that six barbs are configured to extend in a direction orthogonal to the longitudinal axis and disposed about the longitudinal axis proximate the maximal diameter of the second generally conic-like dome.

14. The blood filter of claim 9, in which the barbs extend generally in direction orthogonal to the longitudinal axis with up to 20 degrees in variation from the orthogonal axis.
